(19) United States
(12) Patent Application Publication
(10) Pub. No.: US 2009/0062810 A1
(43) Pub. Date: Mar. 5, 2009

(54) Surgical edged tool

(75) Inventor: Takeshi Sagara, Ube-shi (JP)

Correspondence Address: POSZ LAW GROUP, PLC, 12040 SOUTH LAKES DRIVE, SUITE 101, RESTON, VA, 20191, US

(73) Assignee: KAI R&D CENTER CO., LTD.

(21) Appl. No.: 12/230,715

(22) Filed: Sep. 4, 2008

(30) Foreign Application Priority Data
Sep. 5, 2007 (JP) 2007-229914

(51) Int. Cl. A61F 9/007

(52) U.S. Cl. 606/107

(57) ABSTRACT

A blade plate 2 includes cutting edges 5 on both sides in a lateral direction Y. The cutting edges 5 extend along a longitudinal direction X. Each cutting edge 5 includes an arcuate distal cutting edge 8 and a main cutting edge 10. The distal cutting edges 8 are continuous via a distal joining portion 7. Each main cutting edge 10 extends from the corresponding distal cutting edge 8 via an intermediate joining portion 9 toward the proximal portion of the blade plate 2 to an cutting edge end portion 11. The width W8 between the distal cutting edges 8 of the cutting edges 5 gradually increases from the distal joining portion 7 toward the intermediate joining portions 9. The width W10 between the main cutting edges 10 of the cutting edges 5 gradually increases from the intermediate joining portions 9 toward the cutting edge ends 11. The width W11 between the cutting edge ends 11 of the cutting edges 5 is 2.0 mm or less.

### BACKGROUND OF THE INVENTION

The present invention relates to an edged tool used, for example, in various types of eye surgeries, and more particularly, to an improved ophthalmic edged tool used in a filtering bleb reconstructive surgery performed after trabeculectomy in a glaucoma surgery.

Conventionally, the trabeculectomy has been known as a glaucoma surgery performed to lower a high ocular tension. After the trabeculectomy, the flow of-aqueous humor is blocked due to cicatrix adhesion in a filtering bleb, which reduces the filtering effect. This can raises the ocular tension again. In such a case, a surgery is performed in which the cicatrix underneath conjunctiva and in the filtering bleb is ablated to reconstruct the filtering bleb, so that the ocular tension is prevented from increasing.

In such filtering bleb reconstructive surgery, a syringe need of 23 to 27 gauge (0.64 mm to 0.41 mm in outer diameter) is conventionally used for ablating cicatrix adhesion in the filtering bleb.

The syringe needle has a sharpened tip only at the distal end, and only the sharpened tip contributes to ablation. Thus, in such ablating operation, an operator receives intense resistance when manipulating the syringe needle, which decreases the efficiency. As a result, ablation is insufficiently performed.

In a cataract surgery, a crescent knife (refer to Japanese Patent No. 3066315) is used. This conventional crescent knife has a relatively wide blade plate (typically, not less than 2.6 mm in the width). Thus, such a crescent knife is difficult to handle in the filtering bleb reconstructive surgery in the glaucoma surgery.

### SUMMARY OF THE INVENTION

Accordingly, it is an objective of the present invention to improve the operability of a surgical edged tool used in filtering bleb reconstructive surgery, which is performed after trabeculectomy in various types of eye surgery, particularly in glaucoma surgery.

To achieve the foregoing objective and in accordance with a first aspect of the present invention, a surgical edged tool including an elongaged blade plate and a cutting edge is provided. The elongated blade plate has a distal end, a proximal portion, opposed anterior and posterior surfaces, and opposed longitudinal side edges extending between the distal end and the proximal portion. The cutting edge is formed on each side edge of the blade plate on at least one of the anterior and posterior surfaces. The cutting edges on both side edges are continuously connected via a distal joining portion at the distal end of the blade plate. Each of the cutting edges includes a distal cutting edge and a main cutting edge. The distal cutting edge extends toward the proximal portion of the blade plate from the distal joining portion to an intermediate joining portion. The main cutting edge continuously extends toward the proximal portion of the blade plate from the intermediate joining portion to a cutting edge end portion. A width between both of the distal cutting edges is set so as to increase toward the intermediate joining portions from the distal joining portion. A width between both of the main cutting edges is set so as to increase or to be constant toward the proximal portion of the blade plate from the intermediate joining portion. A width between both of the cutting edge end portions of the cutting edges is set to be less than or equal to 2.0 mm.

In accordance with a second aspect of the present invention, a surgical edged tool including an elongaged blade plate and a cutting edge is provided. The elongated blade plate has a distal end, a proximal portion, opposed anterior and posterior surfaces, and opposed longitudinal side edges extending between the distal end and the proximal portion. The cutting edge is formed on each side edge of the blade plate on at least one of the anterior and posterior surfaces. The cutting edges on both side edges are continuously connected via a distal joining portion at the distal end of the blade plate. Each of the cutting edges includes a distal cutting edge and a main cutting edge. The distal cutting edge extends toward the proximal portion of the blade plate from the distal joining portion to an intermediate joining portion. The main cutting edge continuously extends toward the proximal portion of the blade plate from the intermediate joining portion to a cutting edge end portion. A width between both of the distal cutting edges is set so as to increase toward the intermediate joining portions from the distal joining portion. A width between both of the main cutting edges is set so as to increase or to be constant toward the proximal portion of the blade plate from the intermediate joining portion. The blade plate has a dimension that satisfies following conditions:

L≧2.5 W and T≦0.5 W

where L is a maximum length of the blade plate, W is a maximum width of the blade plate and T is a maximum thickness of the blade plate.

Other aspects and advantages of the present invention will become apparent from the following description, taken in conjunction with the accompanying drawings, illustrating by way of example the principles of the invention.

### BRIEF DESCRIPTION OF THE DRAWINGS

The invention, together with objects and advantages thereof, may best be understood by reference to the following description of the presently preferred embodiments together with the accompanying drawings in which:

FIG. 1(a) is a front view illustrating the entirety of a surgical edged tool according to a first embodiment;

FIG. 1(b) is a side view showing the edged tool of FIG. 1(a);

FIG. 2(a) is an enlarged partial front view showing the blade body of FIG. 1(a);

FIG. 2(b) is a side view showing the blade body of FIG. 2(a);

FIG. 2(c) is a further enlarged partial side view showing the blade body of FIG. 2(b);

FIG. 3(a) is an enlarged partial front view showing the entirety of the blade plate in the blade body of FIG. 2(a);

FIG. 3(b) is a rear view showing the blade plate of FIG. 3(a);

FIG. 3(c) is a further enlarged side view showing the blade body of FIG. 2(c);

FIG. 3(d) is an enlarged partial side view showing a connecting portion of FIG. 3(c);

FIG. 4(a) is an enlarged cross-sectional view taken along line 4a-4a of FIG. 3(a);

FIG. 4(b) is an enlarged cross-sectional view taken along line 4b-4b of FIG. 3(a);

FIG. 4(c) is an enlarged cross-sectional view taken along line 4c-4c of FIG. 3(a);

FIG. 5(a) is an enlarged partial front view a portion of a surgical edged tool according to a second embodiment that corresponds to a portion of the first embodiment shown in FIG. 2(a);

FIG. 5(b) is a side view showing the blade body of FIG. 5(a);

FIG. 5(c) is a further enlarged side view showing the blade body of FIG. 5(b);

FIG. 5(d) is a further enlarged side view showing a connecting portion of FIG. 5(c);

FIG. 6(a) is an enlarged partial front view a portion of a surgical edged tool according to a third embodiment that corresponds to a portion of the first embodiment shown in FIG. 3(a);

FIG. 6(b) is a rear view showing the blade body of FIG. 6(a); and

FIG. 6(c) is a side view showing the blade body of FIG. 6(a).

### DETAILED DESCRIPTION OF THE PREFERRED EMBODIMENTS

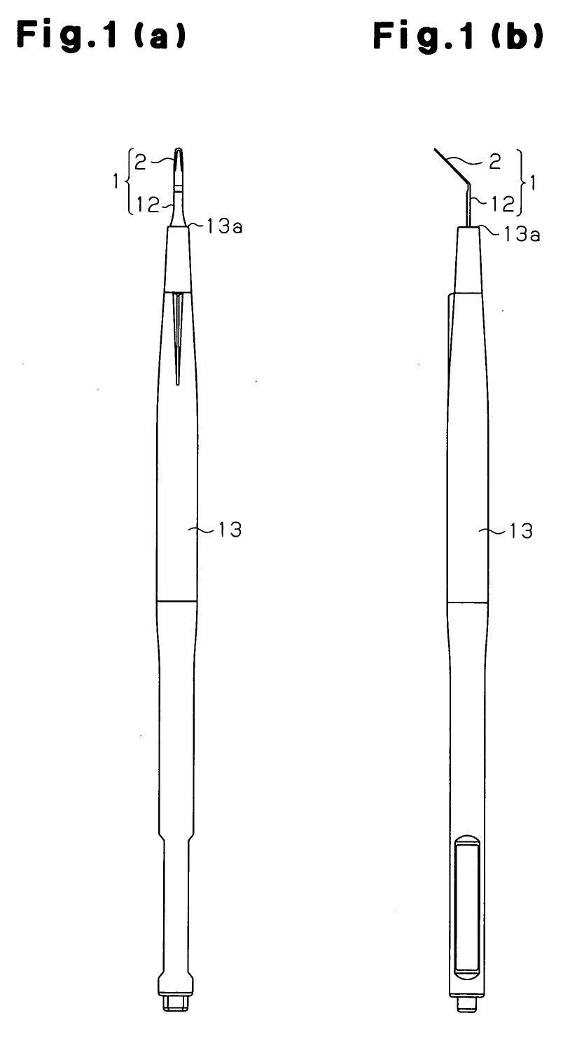

A surgical edged tool according to a first embodiment of the present invention will now be described with reference to FIGS. 1(a) to 4(c).

A surgical edged tool according to the first embodiment shown in FIGS. 1(a) and 1(b) is used particularly in filtering bleb reconstructive surgery performed after trabeculectomy. The edged tool includes a holder 13 and a blade body 1 fixed to a head 13a of the holder 13. The blade body 1 is formed of metal (for example, martensite stainless steel). The blade body 1 also has a blade plate 2 and a blade support 12 supporting the blade plate 2. The blade plate 2 and the blade support 12 are formed integrally.

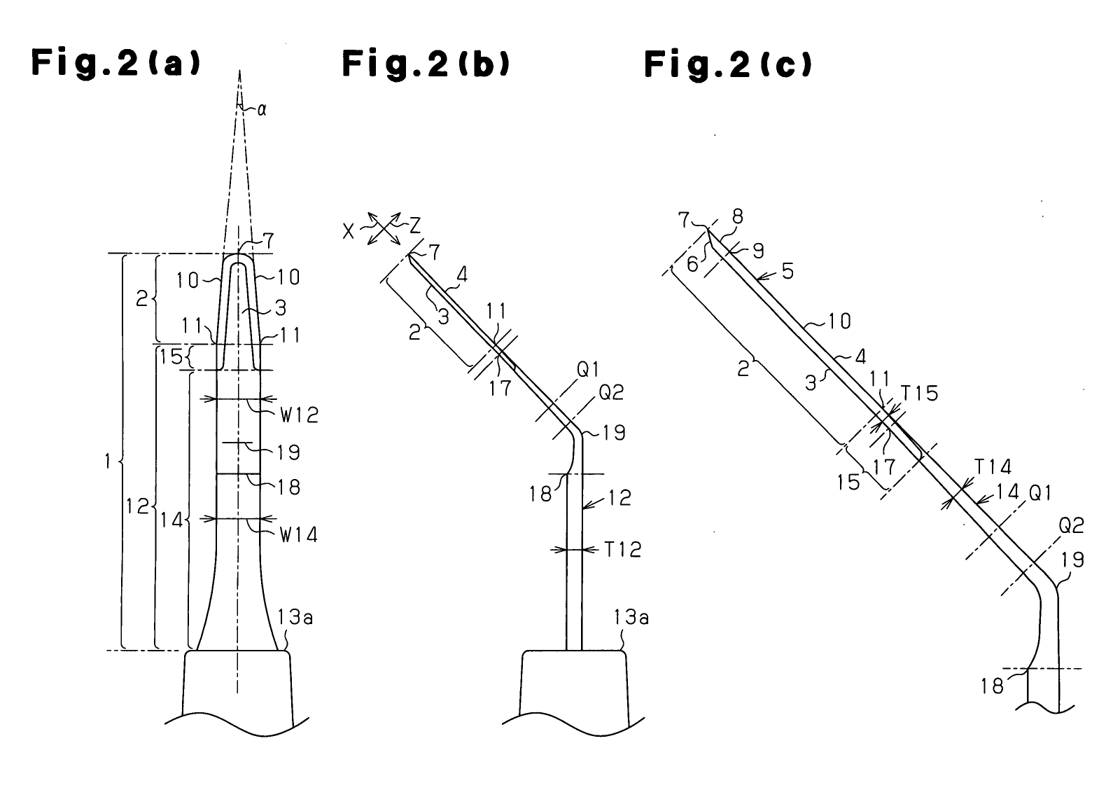

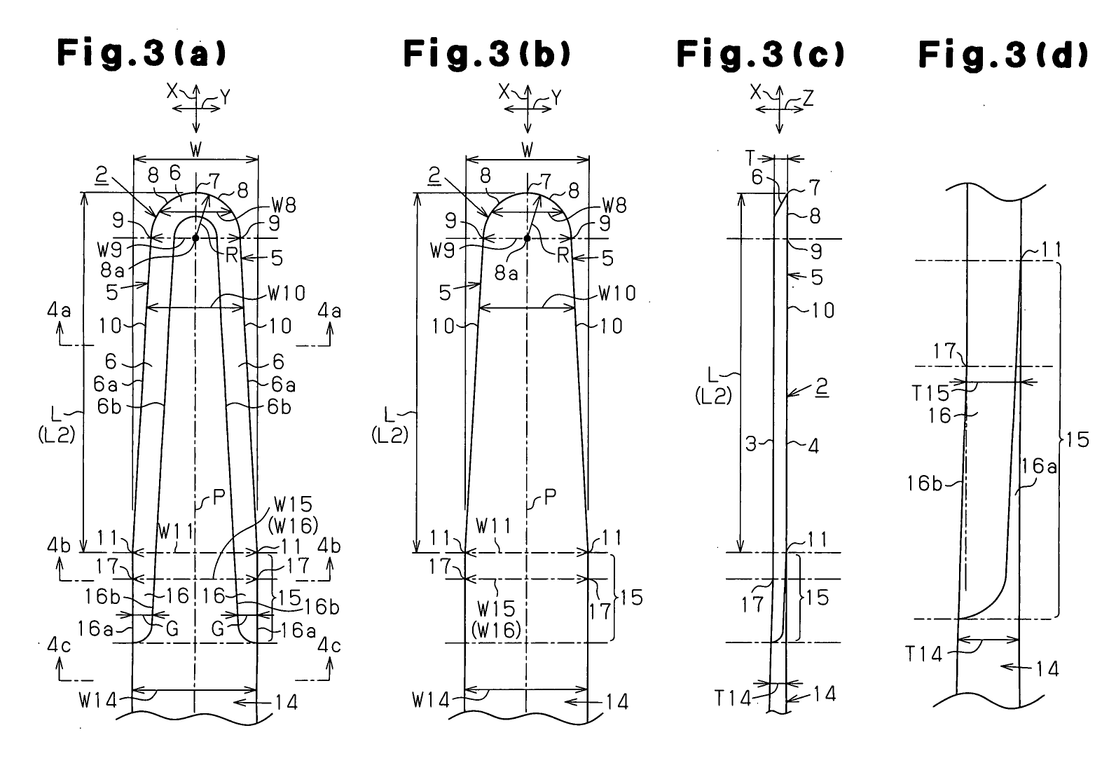

As shown in FIG. 2(b), the blade plate 2 of the blade body 1 has an anterior surface 3 formed on the left side in the direction Z of its thickness and a flat posterior surface 4 formed on the right side in the thickness direction Z. As shown in FIG. 3(a), an inclined bevel 6 is formed on each side of the blade plate 2 in the anterior surface 3. Each bevel 6 is inclined between a sharpened edge 6a and a boundary edge 6b. The bevels 6 are continuously connected to each other at the distal end of the blade plate 2.

Each bevel 6 has a cutting edge 5 extending in a longitudinal direction X in a periphery of the blade plate 2. The cutting edges 5 are continuously connected to each other through a distal joining portion 7 at the distal end of the blade plate 2. Each cutting edge 5 includes a distal cutting edge 8 that extends from the distal joining portion 7 toward the proximal portion of the blade plate 2. Each cutting edge 5 includes a main cutting edge 10 that extends from the distal cutting edge 8 toward the proximal portion of the blade plate 2 and to the cutting edge end portion 11 via an intermediate joining portion 9. As described above, the distal joining portion 7, the distal cutting edge 8, the intermediate joining portion 9, the main cutting edge 10, and the cutting edge end portion 11, which form each cutting edge 5, are formed continuously so as to be continuous at the sharpened edge 6a of the bevel 6.

The cutting edges 5 are symmetric with respect to a center line P of the blade plate 2. Curves connecting the intermediate joining portions 9 and the distal cutting edges 8 to the distal joining portion 7 form a single arc of a radius R. The center of the arc is referred to as a center 8a and located on the center line P of the blade plate 2. The arc bulges to the outside of the distal end of the blade plate 2. The width W8 between the distal cutting edges 8 of the cutting edges 5 in a lateral direction Y gradually increases from the distal joining portion 7 toward the intermediate joining portions 9. The width W10 between the main cutting edges 10 of the cutting edges 5 in the lateral direction Y gradually increases from the intermediate joining portions 9 toward the cutting edge ends 11.

As shown in FIGS. 2(a) and 2(c), the blade support 12 of the blade body 1 has an arm 14 attached to the head 13a of the holder 13, and a connecting portion 15 that is continuously formed with the blade plate 2 at the cutting edge ends 11 of the cutting edges 5. The back side of the connecting portion 15, the back side of the arm 14, and the posterior surface 4 of the blade plate 2 are continuous flat surfaces.

As shown in FIG. 3(a), a cutaway portion 16 is formed on each side of the front side of the connecting portion 15. Each cutaway portion 16 is continuous to the bevel 6 of the blade plate 2, and extends toward the head 13a and to the arm 14. Each cutaway portion 16 is formed between a linear edge 16a (not a cutting edge), which is continuous to the sharpened edge 6a of the bevel 6, and a boundary edge 16b formed, which is continuous to the boundary edge 6b of the bevel 6. Each cutaway portion 16 is also inclined relative to the bevel 6.

The width W15 of the connecting portion 15, that is, the width W16 between the edges 16a of the cutaway portions 16 is equal to the width W11 between the cutting edge ends 11 of the cutting edges 5 in a range from the cutting edge ends 11 of the cutting edges 5 to the arm 14. In each cutaway portion 16, the width G between the edge 16a and the boundary edge 16b along the edge 16a is set to gradually decrease from the cutting edge end portion 11 of the cutting edge 5 toward the head 13a of the holder 13, and becomes zero in the vicinity of the arm 14.

As shown in FIG. 3(d), the connecting portion 15 includes an inclined portion 17, and the thickness T15 of the connecting portion 15 increases toward the head 13a of the holder 13. The inclined portion 17 is closer to the head 13a of the holder 13 than the cutting edge end portion 11 of the cutting edge 5 is. The thickness T15 of the connecting portion 15 is constant from the cutting edge end portion 11 of the cutting edge 5 to the inclined portion 17, and increases from the inclined portion 17 toward the arm 14.

As shown in FIG. 2(a), the width W14 of the arm 14 is equal to the width W15 of the connecting portion 15 in the vicinity of the connecting portion 15, but increases in the vicinity of the head 13a of the holder 13. As shown in FIG. 2(b), the arm 14 includes a thick portion 18, the thickness T14 of which increases toward the head 13a of the holder 13 and then becomes constant. The thick portion 18 is located between the inclined portion 17 and the head 13a of the holder 13. The thickness T12 of the blade support 12 extending from the connecting portion 15 to the arm 14 gradually increases from the inclined portion 17 to the thick portion 18.

Between the thick portion 18 and the cutting edge ends 11 of the cutting edges 5, a first center portion Q1 is located at a middle position along the blade support 12. Between the inclined portion 17 and the thick portion 18, a bent portion 19 is located closer to the head 13a of the holder 13 than the first center portion Q1 is. Alternatively, between the distal joining portion 7 of the distal cutting edges 8 of the blade plate 2 and the head 13a of the holder 13, a second center portion Q2 is located at a middle position along the blade plate 2 and the blade support 12. Between the inclined portion 17 and the head 13a of the holder 13, the bent portion 19 is located closer to the head 13a of the holder 13 than the second center portion Q2 is. The blade support 12 extends from the head 13a of the holder 13 along the holder 13, and is bent at the bent portion 19. A portion of the blade body 1 between the bent portion 19 and the distal joining portion 7 extends along the longitudinal direction X, and is bent with respect to the axial direction of the holder 13. The bent angle of the bent portion 19 is 45° relative to the axial direction of the holder 13, but may be any angle between 15° and 60°.

The micro-Vickers hardness of the blade body 1, which is made of martensite stainless steel, is set to HV550 to HV650 to achieve a good balance between the strength of the cutting edges 5 and the flexibility of the bent portion 19 of the blade support 12.

The dimensions of the blade body 1 of the edged tool according to the first embodiment are set as shown below.

The width W11 between the cutting edge ends 11 of the cutting edges 5 is less than or equal to 2.0 mm.

The width W9 (FIG. 3(a)) between the intermediate joining portions 9 of the cutting edges 5 is greater than or equal to 0.4 mm and less than or equal to 1.8 mm, preferably greater than or equal to 0.6 mm and less than or equal to 1.4 mm, and more preferably greater than or equal to 0.6 mm and less than or equal to 1.0 mm. The width W11 between the cutting edge ends 11 of the cutting edges 5 is greater than or equal to 0.6 mm and less than or equal to 2.0 mm, preferably greater than or equal to 0.8 mm and less than or equal to 1.6 mm, and more preferably greater than or equal to 0.8 mm and less than or equal to 1.2 mm.

The width W11 between the cutting edge ends 11 of the cutting edges 5 is 1.1 to 1.5 times greater than the width W9 between the intermediate joining portions 9 of the cutting edges 5, and preferably 1.2 to 1.4 times greater than the width W9.

When the maximum length, the maximum width, and the maximum thickness of the blade plate 2 are represented by L (FIG. 3(a)), W (W11 in FIG. 3(a)), and T (FIG. 3(c)), respectively, the maximum length L, the maximum width W, and the maximum thickness T are set to meet the following conditions.

L≧2.5 W and T≦0.5 W

preferable condition: 2.5 W≦L≦6 W and 0.05 W≦T≦0.4 W

The length L2 (FIG. 3(a)) from the distal joining portion 7 of the cutting edges 5 to the cutting edge ends 11 of the cutting edges 5 is greater than or equal to 2.0 mm and less than or equal to 8.0 mm, and preferably greater than or equal to 2.5 mm and less than or equal to 6.5 mm. The length L2 from the distal joining portion 7 of the cutting edges 5 to the cutting edge ends 11 of the cutting edges 5 is two to ten times greater than the width W9 between the intermediate joining portions 9 of the cutting edges 5, and preferably two to six times greater than the width W9.

The width W12 (FIG. 2(a)) of the blade support 12 is between one time and four times the width W11 between the cutting edge ends 11 of the cutting edges 5, and preferably one to two times greater than the width W11.

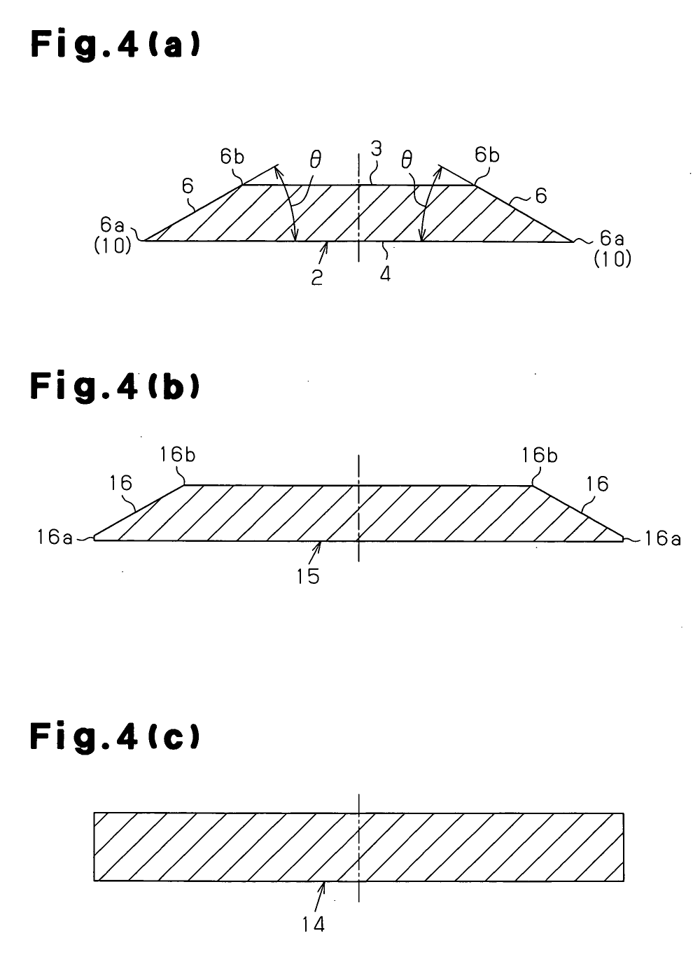

The sharpening angle θ (FIG. 4(a)) of the cutting edges 5 is greater than or equal to 10° and less than or equal to 60°, and preferably greater than or equal 25° and less than or equal to 45°. If the sharpening angle θ is excessively acute, the rigidity of the blade plate 2 is reduced. In contrast, if the sharpening angle θ is excessively obtuse, the resistance at ablation and incision increases. After various kinds of trials, it has been found out that a sharpening angle θ greater than or equal to 25° and less than or equal to 45° achieves a high rigidity and a low resistance.

The intersection angle α (FIG. 2(a)) defined by the main cutting edges 10 of the cutting edges 5 is greater than or equal to 0.5° and less than or equal to 15°, and preferably greater than or equal to 1° and less than or equal to 10°.

The distal cutting edge 8 between the distal joining portion 7 and the intermediate joining portion 9 is formed along an arc having a predetermined radius R, and the radius R is greater than or equal to 0.2 mm and less than or equal to 0.9 mm, and preferably greater than or equal to 0.3 mm and less than or equal to 0.7 mm.

The edged tool according to the first embodiment has the following advantages.

(1) Since the width W8 between the distal cutting edges 8 gradually increases from the distal joining portion 7 toward the intermediate joining portions 9, a portion of the blade plate 2 is narrow in the vicinity of the distal joining portion 7. Thus, during surgery, the blade plate 2 is easily inserted into an incision site.

(2) The width W10 between the main cutting edges 10 gradually increases from the intermediate joining portions 9 toward the cutting edge ends 11. Thus, after the distal cutting edges 8 are inserted into a site of surgery, the main cutting edges 10 can be further inserted with a small resistance.

(3) Since each cutting edge 5 has the distal cutting edge 8 and the main cutting edge 10, not only the distal cutting edge 8 but also the main cutting edge 10 are effectively used. Thus, ablation and incision of cicatrix adhesion in a filtering bleb and its surroundings can be performed with a low resistance.

(4) The width W11 between the cutting edge ends 11 of the cutting edges 5 is 2.0 mm or less. In typical trabeculectomy, a filtering bleb of 4 mm in diameter is formed. Thus, if the width W of the blade plate 2 is less than half the width of the filtering bleb or less, surgery in narrow sections is facilitated. Therefore, ablation and incision of cicatrix adhesion in a filtering bleb and its surroundings is facilitated.

(5) Since the width W of the blade plate 2 is as small as 2.0 mm, the insertion opening (cut) formed by the blade plate 2 is small. Thus, minimum ablation and incision is performed on a necessary section, and excessive cutting is prevented. This reduces the patient burden and reliably prevents infections.

(6) The maximum length L, the maximum width W, and the thickness T of the blade plate 2 are set to meet the following conditions.

L≧2.5 W and T≦0.5 W

Due to these dimensions, the blade plate 2 is thin and elongated. This not only facilitates ablation and incision of cicatrix adhesion, but also facilitates treatment to a site away from the insertion opening (cut) formed by the blade plate 2. Further, minimum ablation and incision is performed on a necessary section while maintaining the cut to be small, and excessive cutting is prevented. This reduces the patient burden and reliably prevents infections.

(7) The blade plate 2 is fixed to the head 13a of the holder 13 by means of the blade support 12, which has the connecting portion 15 continuous to the cutting edges 5 at the cutting edge ends 11. The connecting portion 15 has the inclined portion 17, the thickness T15 of which increases toward the head 13a of the holder 13. The inclined portion 17 is closer to the head 13a of the holder 13 than the cutting edge ends 11 of the cutting edges 5 are.

Therefore, even if the blade plate 2 is made thin, the inclined portion 17 maintains the rigidity of the blade support 12. As a result, the blade support 12 is prevented from being flexed. This facilitates ablation and incision of a filtering bleb and its surroundings.

(8) The blade support 12 has the thick portion 18 in which the thickness of the blade support 12 increases toward the head 13a of the holder 13 and then becomes constant. The thick portion 18 is located between the inclined portion 17 and the head 13a of the holder 13. Between the thick portion 18 and the cutting edge ends 11 of the cutting edges 5, the first center portion Q1 is located at a middle position along the blade support 12. The bent portion 19, which is located between the inclined portion 17 and the thick portion 18, is closer to the head 13a of the holder 13 than the first center portion Q1 is.

The bent portion 19 in the blade support 12 facilitates the manipulation of the edged tool. The edged tool can be inserted up to a section near the bent portion 19, a large amount of insertion is ensured. A site away from the insertion opening (cut) is also easily treated.

(9) The maximum length L, the maximum width W, and the maximum thickness T of the blade plate 2 are set as follows.

L≧2.5 W and T≦0.5 W

Due to such dimensions of the blade plate 2, the blade plate 2 is thin and elongated. Therefore, not only ablation and incision of cicatrix adhesion of a filtering bleb and its surroundings is facilitated, but also, treatment of a site away from the insertion opening (cut) formed by the blade plate 2 is facilitated. Also, while maintaining the cut to be small, only a necessary site is ablated and incised. Excessive cutting is prevented. This reduces the patient burden and reliably prevents infections.

(10) Between the distal joining portion 7 of the distal cutting edges 8 of the blade plate 2 and the head 13a of the holder 13, the second center portion Q2 is located at a middle position of the distance in a direction along the blade plate 2 and the blade support 12 extend. The bent portion 19, which is located between the inclined portion 17 and the head 13a of the holder 13, is closer to the head 13a of the holder 13 than the second center portion Q2 is.

Since the bent portion 19 is provided in the blade support 12, the edged tool is easy to manipulate. The edged tool can be inserted up to a section near the bent portion 19, a large amount of insertion is ensured. A site away from the insertion opening (cut) is thus also easily treated.

(11) The distal cutting edges 8 are arcuately bulged outward of the distal end in the longitudinal direction X of the blade plate 2.

This prevents the blade plate 2 from being excessively pierced.

(12) The width W9 between the intermediate joining portions 9 of the cutting edges 5 is greater than or equal to 0.4 mm and less than or equal to 1.8 mm. The width W11 between the cutting edge ends 11 of the cutting edges 5 is greater than or equal to 0.6 mm and less than or equal to 2.0 mm.

Considering the fact that a typical filtering bleb is approximately 4 mm in diameter, the dimensions of the blade plate 2 are suitable for ablating and incising cicatrix adhesion in a filtering bleb and its surroundings. Also, the cut formed by the blade plate 2 is relatively small. This reduces the patient burden and reliably prevents infections.

(13) The width W11 between the cutting edge ends 11 of the cutting edges 5 is 1.1 to 1.5 times greater than the width W9 between the intermediate joining portions 9 of the cutting edges 5.

The main cutting edges 10 are inclined. Thus, after the distal cutting edges 8 of a narrow width are inserted into an objective site, the main cutting edges 10 can be further inserted with a small resistance. This reduces the size of the cut formed by the blade plate 2. The blade plate 2 thus reduces the patient burden and reliably prevents infections.

(14) The cutting edges 5 are formed to be line symmetric with respect to the center line P of the width of the blade plate 2.

Therefore, since resistance is evenly generated in the lateral direction Y of the blade plate 2, ablation and incision of cicatrix adhesion in a filtering bleb and its surroundings is easily performed.

(15) The maximum length L from the distal joining portion 7 of the cutting edges 5 to the cutting edge ends 11 of the cutting edges 5 is greater than or equal to 2.0 mm and less than or equal to 8.0 mm. The maximum length L from the distal joining portion 7 of the cutting edges 5 to the cutting edge ends 11 of the cutting edges 5 is two to ten times greater than the width W9 between the intermediate joining portions 9 of the cutting edges 5.

Due to these dimensions, the blade plate 2 having a predetermined width is thin and elongated. Therefore, ablation and cutting of cicatrix adhesion in a filtering bleb and its surroundings is facilitated.

(16) The blade support 12, which continues to the cutting edge ends 11 of the cutting edges 5, is fixed to the head 13a of the holder 13. The width W12 of the blade support 12 is one to four times greater than the width W11 between the cutting edge ends 11 of the cutting edges 5.

Therefore, even if the width of the blade plate 2 is narrow, a sufficient rigidity of the blade support 12 is ensured, and the sight of the operator is not blocked during surgery.

(17) The blade support 12 having the connecting portion 15, which continues to the cutting edge ends 11 of the cutting edges 5, is fixed to the head 13a of the holder 13. The width W15 of the connecting portion 15 is equal to the width W11 between the cutting edge ends 11 of the cutting edges 5.

Thus, even if the edged tool is inserted up to the connecting portion 15 of the blade support 12, the insertion opening (cut) is not widened but is maintained. Also, the rigidity of the connecting portion 15, which supports the blade plate 2, is ensured.

(18) The bevels 6 for forming the cutting edges 5 are formed on at least one of the anterior surface 3 and the posterior surface 4 of the blade plate 2. In the sharpened edge 6a of each bevel 6, the distal joining portion 7, the distal cutting edge 8, the intermediate joining portion 9, the main cutting edge 10, and the cutting edge end portion 11, which form the cutting edge 5, are formed continuously. On both sides of the blade support 12 in the lateral direction Y, the cutaway portions 16 continuous to the bevels 6 of the blade plate 2 extend toward the head 13a of the holder 13.

Therefore, since the cutaway portions 16 that continue from the bevels 6 of the blade plate 2 are provided in the blade support 12, the edged tool can be pierced further from the blade plate 2 up to the blade support 12 having the cutaway portions 16 with a small resistance.

(19) The width W16 between the edges 16a of the cutaway portions 16 in the blade support 12 is constant.

Thus, even if the edged tool is inserted up to the blade support 12, the insertion opening (cut) is not widened but is maintained.

(20) In each cutaway portion 16, the width G between the edge 16a and the boundary edge 16b along the edge 16a is set to gradually decrease from the cutting edge end portion 11 of the cutting edge 5 toward the head 13a of the holder 13.

Since the width G of the cutaway portion 16 is set as described above, the piercing resistance is not abruptly increased even if the edged tool is inserted up to the blade support 12.

(21) The sharpening angle θ of the cutting edges 5 is greater than or equal to 10° and less than or equal to 60°.

Thus, ablation and incision of cicatrix adhesion in a filtering bleb and its surroundings can be performed with a low resistance.

(22) The intersection angle α defined by the main cutting edges 10 of the cutting edges 5 is greater than or equal to 0.5° and less than or equal to 15°.

Therefore, since the main cutting edges 10 are inclined, the main cutting edges 10 can be pierced with a small resistance after the distal cutting edges 8 having a narrow width are inserted into an objective site of surgery. Also, since the cut formed by the blade plate 2 is relatively small, the patient burden is reduced and infections are prevented.

(23) The distal cutting edge 8 between the distal joining portion 7 and the intermediate joining portion 9 in each cutting edge 5 is formed along an arc having a predetermined radius R, which is greater than or equal to 0.2 mm and less than or equal to 0.9 mm.

Therefore, since the radius R of the distal cutting edges 8 is relatively small, the distal cutting edges 8 can be finely manipulated. Therefore, ablation and cutting of cicatrix adhesion in a filtering bleb and its surroundings is delicately performed.

As described above, if the ophthalmic edged tool according to the present embodiment is used in filtering bleb reconstructive surgery after trabeculectomy in glaucoma surgery, ablation and incision of cicatrix adhesion in a filtering bleb and its surroundings is facilitated. Therefore, in filtering bleb reconstructive surgery, the operability of the edged tool is improved, and the patient burden is reduced.

A number of trial products according to the present invention have been made. A first trial produce (L=4.0 mm, W9=0.8 mm, W11=1.1 mm, α=4.8°, R=0.4 mm, T=0.15 mm, T12=0.5 mm, θ=30°), and a second trial product (L=4.0 mm, W9=1.0 mm, W11=1.4 mm, α=6.5°, R=0.5 mm, T=0.15 mm, T12=0.5 mm, θ=30°) were actually used in filtering bleb reconstructive surgery. As a result, when piercing a conjunctiva with the blade plate 2, the operator felt no great resistance. The blade plate 2 was smoothly inserted with a small resistance into a site where cicatrix adhesion was considered to be intense. The resistance of lateral swing of the blade plate 2 was significantly less than the case where a syringe needle was used. It was confirmed that the edged tool had the above described advantages.

Generally, if cicatrix adhesion excessively develops after trabeculectomy, the cicatrix adhesion can hardly be ablated in a filtering bleb and its surroundings by using a conventional syringe needle. Thus, normally, filtering bleb reconstructive surgery is performed within three months after trabeculectomy. However, when the ophthalmic edged tool according to the present embodiment was used, filtering bleb reconstructive surgery was successful after eighteen months from trabeculectomy.

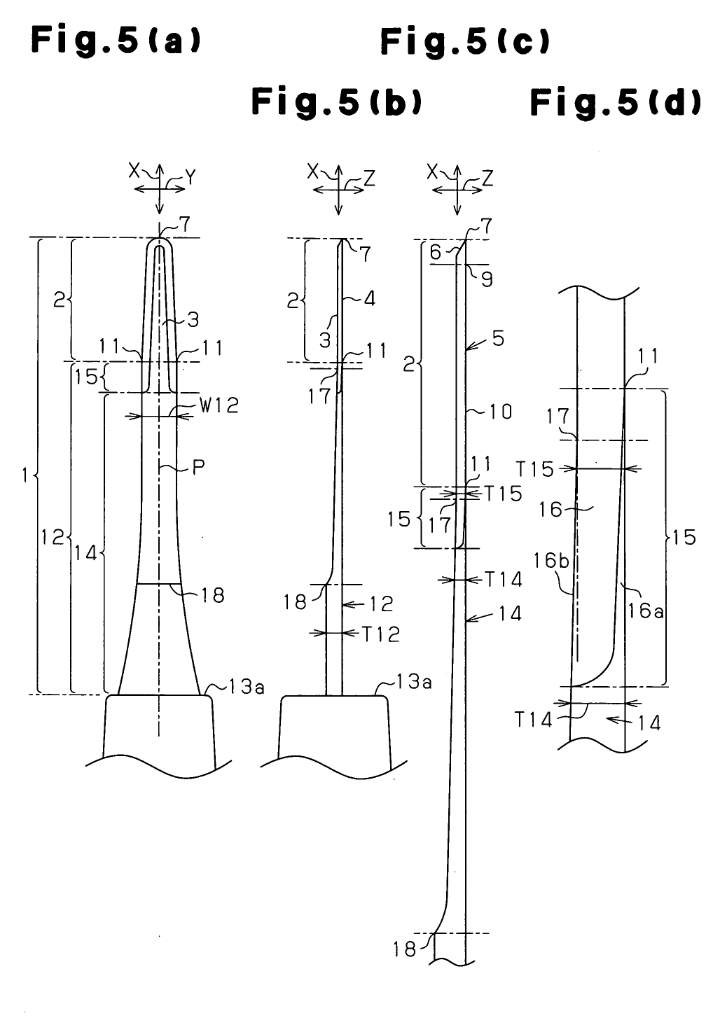

FIGS. 5(a) to 5(c) show a surgical edged tool according to a second embodiment. The surgical edged tool according to the second embodiment is different from the first embodiment mainly in that the bent portion 19 is omitted from the arm 14 of the blade support 12, and the blade body 1 formed by the blade support 12 and the blade plate 2 extends linearly in a direction along which the holder 13 extends. The edged tool according to the second embodiment has the same advantages as the edged tool according to the first embodiment.

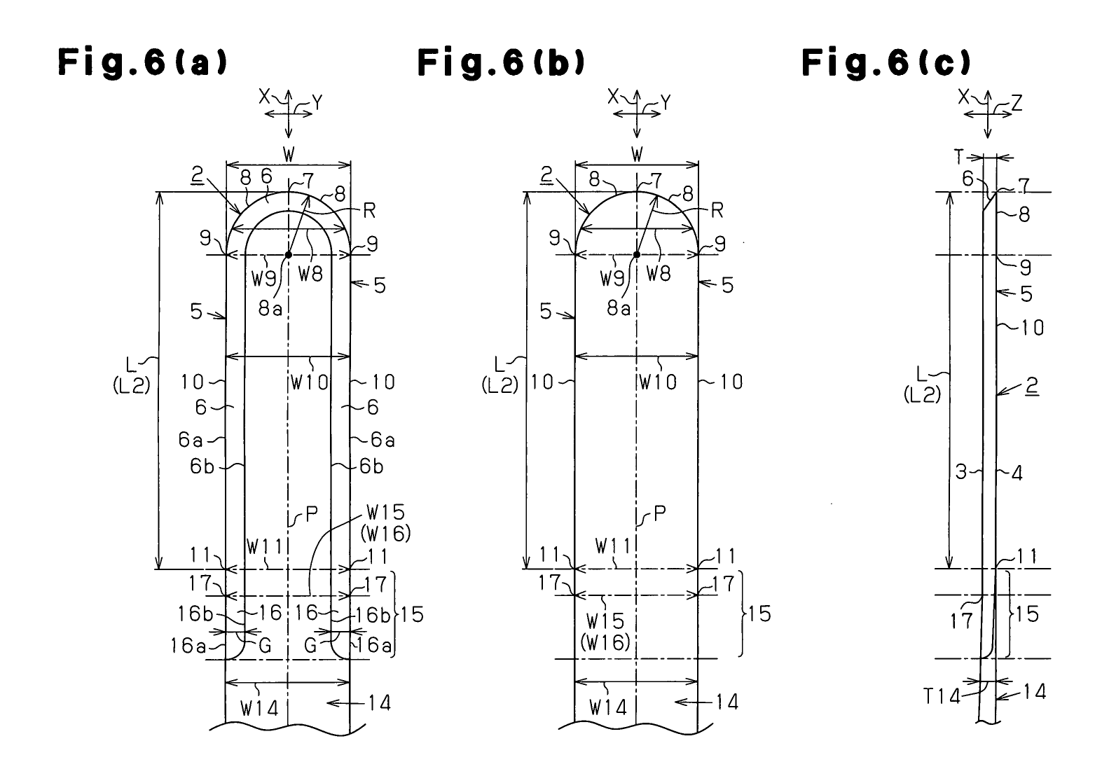

FIGS. 6(a) to 6(c) show a surgical edged tool according to a third embodiment. The surgical edged tool according to the third embodiment is different from the first embodiment mainly in that the width W10 of the main cutting edges 10 of the cutting edges 5 is set to be constant between the intermediate joining portions 9 and the cutting edge ends 11. Therefore, in each cutaway portion 16 of the connecting portion 15, the width G between the edge 16a and the boundary edge 16b extending along the edge 16a has a constant section between the cutting edge end portion 11 of the cutting edge 5 and the arm 14, and becomes zero in the vicinity of the arm 14. The edged tool according to the third embodiment has the same advantages as the edged tool according to the first embodiment.

The edged tools according to the first to third embodiments may be modified as follows.

In each of the edged tools according to the first to third embodiments, the intermediate joining portions 9 of the cutting edges 5 of the blade plate 2 only form boundaries between the distal cutting edges 8 and the main cutting edges 10. Instead of this configuration, the intermediate joining portions 9 may be intermediate cutting edges that extend between the distal cutting edges 8 and the main cutting edges 10 to connect the cutting edges 8, 10 to each other. The width between the intermediate cutting edges of the cutting edges 5 may be constant or may be set to gradually increase from the distal cutting edges 8 to the main cutting edges 10. Likewise, the distal joining portion 7 may be a curved or straight cutting edge that extends between the distal cutting edges 8 to connect the distal cutting edges 8 to each other.

Further, the distal cutting edges 8 of the cutting edges 5 do not need to be curved as an arc, but may extend linearly.

Further, the main cutting edges 10 of the cutting edges 5 may be curved instead of being linear.

A single or multiple recesses may be formed on the anterior surface 3 or the posterior surface 4 of the blade plate 2. This reduces the contact area of the blade plate, and thus lowers the piercing resistance.

The blade plate 2 may be arcuately curved in the longitudinal direction X, in the lateral direction Y, or in both of the longitudinal direction X and eth lateral direction Y. Further, a part of the blade support 12 that continues from the blade plate 2 may be curved arcuately. The radius of curvature of the arcuate portion is preferably close to the radius of the eyeball, and, for example, greater than or equal to 6 mm and less than or equal to 20 mm.

In each of the edged tools according to the first to third embodiments, the bevels 6, or the cutting edges 5, are formed only on the anterior surface 3 of the blade plate 2. However, only the posterior surface 4 of the blade plate 2 may be provided with bevels 6, or cutting edges. Further, both of the anterior surface 3 and the posterior surface 4 may be provided with bevels 6, or the cutting edges.

The color of the holder 13 of the edged tool is preferably a color that contrasts favorably with the color of the drape used in surgery. Since the color of a drape is often light blue or green, the color of the holder 13 may be yellow, orange, brown, or navy blue.

The ophthalmic edged tools according to the first to third embodiments may be used to cut a conjunctiva in trabeculectomy. The edged tool according to the present invention may be used for purposes other than ophthalmic surgery.

The outer surface of the blade plate 2 of the edged tool may be coated with silicone resin or fluorine resin. This reduces the resistance during use.

Therefore, the present examples and embodiments are to be considered as illustrative and not restrictive and the invention is not to be limited to the details given herein, but may be modified within the scope and equivalence of the appended claims.

What is claimed is:

1. A surgical edged tool comprising:
an elongated blade plate having a distal end, a proximal portion, opposed anterior and posterior surfaces, and opposed longitudinal side edges extending between the distal end and the proximal portion; and
a cutting edge formed on each side edge of the blade plate on at least one of the anterior and posterior surfaces, the cutting edges on both side edges continuously connected via a distal joining portion at the distal end of the blade plate;
wherein each of the cutting edges includes:
a distal cutting edge extending toward the proximal portion of the blade plate from the distal joining portion to an intermediate joining portion; and
a main cutting edge continuously extending toward the proximal portion of the blade plate from the intermediate joining portion to a cutting edge end portion,
wherein a width between both of the distal cutting edges is set so as to increase toward the intermediate joining portions from the distal joining portion,
wherein a width between both of the main cutting edges is set so as to increase or to be constant toward the proximal portion of the blade plate from the intermediate joining portion, and
wherein a width between both of the cutting edge end portions of the cutting edges is set to be less than or equal to 2.0 mm.

2. The surgical edged tool according to claim 1, further comprising:
a connecting portion continuously extending from the proximal portion of the blade plate;
an elongated blade support continuously extending from the connecting portion; and
a holder having a head, on which the blade support is secured,
wherein the connecting portion includes an inclined portion of which the thickness increases toward the head of the holder, and the inclined portion is located at a position closer to the head of the holder than the cutting edge end portions of the cutting edges are.

3. The surgical edged tool according to claim 2, wherein the blade support includes:
a thick portion arranged between the inclined portion and the head of the holder, the thick portion having a shape of which the width becomes thicker and then becomes constant toward the head of the holder; and
a bent portion arranged between the inclined portion and the thick portion,
wherein the thick portion is located at a position closer to the head of the holder than a center portion between the thick portion and the cutting edge end portions of the cutting edges along the blade support is.

4. The surgical edged tool according to claim 1, wherein the blade plate has a dimension that satisfies following conditions:
L≧2.5 W and T≦0.5 W
where L is a maximum length of the blade plate, W is a maximum width of the blade plate, and T is a maximum thickness of the blade plate.

5. The surgical edged tool according to claim 2, wherein the blade support includes a bent portion arranged between the inclined portion and the head of the holder,
wherein the thick portion is located at a position closer to the head of the holder than a center portion between the distal joining portion of the distal cutting edges and the head of the holder along a path of the blade plate and the blade support is.

6. The surgical edged tool according to claim 1, wherein each of the distal cutting edges extends outwardly at the distal end of the blade plate.

7. The surgical edged tool according to claim 1, wherein a width between both of the intermediate joining portions of the cutting edges is greater than or equal to 0.4 mm and less than or equal to 1.8 mm, and a width between both of the cutting edge end portions of the cutting edges is greater than or equal to 0.6 mm and less than or equal to 2.0 mm.

8. The surgical edged tool according to claim 1, wherein a width between both of the cutting edge end portions of the cutting edges is 1.1 to 1.5 times greater than a width between both of the intermediate joining portions of the cutting edges.

9. The surgical edged tool according to claim 1, wherein the cutting edges are line symmetric with respect to a width centerline of the blade plate.

10. The surgical edged tool according to claim 1, wherein a length in longitudinal direction between the distal joining portion and the cutting edge end portions of the cutting edges is greater than or equal to 2.0 mm and less than or equal to 8.0 mm, and a length in longitudinal direction between the distal joining portion and the cutting edge end portions of the cutting edges is two to ten times greater than a width between both of the intermediate joining portions of the cutting edges.

11. The surgical edged tool according to claim 1, further comprising:
a blade support continuously extending from the proximal portion of the blade plate; and
a holder having a head, on which the blade support is secured,
wherein a width of the blade support is 1 to 4 times greater than a width between both of the cutting edge end portions of the cutting edges.

12. The surgical edged tool according to claim 1, further comprising:
a connecting portion continuously extending from the proximal portion of the blade plate;
an elongated blade support continuously extending from the connecting portion; and
a holder having a head, on which the blade support is secured,
wherein a width of the connecting portion is the same as a width between both of the cutting edge end portions of the cutting edges.

13. The surgical edged tool according to claim 1, further comprising:
a blade support continuously extending from the proximal portion of the blade plate;
a holder having a head, on which the blade support is secured; and
a bevel formed on at least one of the anterior and posterior surfaces for forming the cutting edges,
wherein the bevel includes a sharp edge, which is continuously formed by the distal joining portion, the proximal cutting edges, the intermediate joining portions, the main cutting edges, and the cutting edge end portions of the cutting edges, and
wherein a cutaway portion is formed on both sides of the blade support, and each of the cutaway portions continuously extends from each of the bevels toward the head of the holder.

14. The surgical edged tool according to claim 13, wherein each of the cutaway portions includes a side edge, and a width between both of the side edges of cutaway portions is constant.

15. The surgical edged tool according to claim 14, wherein each of the cutaway portions is defined by the side edge and a boundary edge extending substantially parallel with the side edge, and a width between each of the side edges and each of the boundary edges is set so as to decrease toward the head of the holder.

16. The surgical edged tool according to claim 1, wherein a sharpening angle of the main cutting edges is greater than or equal to 10 degrees and less than or equal to 60 degrees.

17. The surgical edged tool according to claim 1, wherein an intersection angle defined by imaginary lines extending along each of the main cutting edges is greater than or equal to 0.5 degrees and less than or equal to 15 degrees.

18. The surgical edged tool according to claim 1, wherein each of the distal cutting edges between the distal joining portion and the intermediate joining portion is an arcuate shape having a diameter greater than or equal to 0.2 mm and less than or equal to 0.9 mm.

19. A surgical edged tool comprising:
an elongated blade plate having a distal end, a proximal portion, opposed anterior and posterior surfaces, and opposed longitudinal side edges extending between the distal end and the proximal portion; and
a cutting edge formed on each side edge of the blade plate on at least one of the anterior and posterior surfaces, the cutting edges on both sides continuously connected via a distal joining portion at the distal end of the blade plate;
wherein each of the cutting edges includes:
a distal cutting edge extending toward the proximal portion of the blade plate from the distal joining portion to an intermediate joining portion; and
a main cutting edge continuously extending toward the proximal portion of the blade plate from the intermediate joining portion to a cutting edge end portion,
wherein a width between both of the distal cutting edges is set so as to increase toward the intermediate joining portions from the distal joining portion;
wherein a width between both of the main cutting edges is set so as to increase or to be constant toward the proximal portion of the blade plate from the intermediate joining portion; and
wherein the blade plate has a dimension that satisfies following conditions:
L≧2.5 W and T≦0.5 W
where L is a maximum length of the blade plate, W is a maximum width of the blade plate and T is a maximum thickness of the blade plate.

20. The surgical edged tool according to claim 19, wherein each of the distal cutting edges extends outwardly at the distal end of the blade plate.

21. The surgical edged tool according to claim 19, wherein a width between both of the intermediate joining portions of the cutting edges is greater than or equal to 0.4 mm and less than or equal to 1.8 mm, and a width between both of the cutting edge end portions of the cutting edges is greater than or equal to 0.6 mm and less than or equal to 2.0 mm.

22. The surgical edged tool according to claim 19, wherein a width between both of the cutting edge end portions of the cutting edges is 1.1 to 1.5 times greater than a width between both of the intermediate joining portions of the cutting edges.

23. The surgical edged tool according to claim 19, wherein the cutting edges are line symmetric with respect to a width centerline of the blade plate.

24. The surgical edged tool according to claim 19, wherein a length in longitudinal direction between the distal joining portion and the cutting edge end portions of the cutting edges is greater than or equal to 2.0 mm and less than or equal to 8.0 mm, and a length in longitudinal direction between the distal joining portion and the cutting edge end portions of the cutting edges is two to ten times greater than a width between both of the intermediate joining portions of the cutting edges.

25. The surgical edged tool according to claim 19, wherein a sharpening angle of the main cutting edges is greater than or equal to 10 degrees and less than or equal to 60 degrees.

26. The surgical edged tool according to claim 19, wherein an intersection angle defined by imaginary lines extending along each of the main cutting edges is greater than or equal to 0.5 degrees and less than or equal to 15 degrees.

27. The surgical edged tool according to claim 19, wherein each of the distal cutting edges between the distal joining portion and the intermediate joining portion is an arcuate shape having a diameter greater than or equal to 0.2 mm and less than or equal to 0.9 mm.
